(19) United States
(12) Patent Application Publication
(10) Pub. No.: US 2024/0006018 A1
(43) Pub. Date: Jan. 4, 2024

(54) METHOD

(71) Applicant: CAMBRIDGE ENTERPRISE LIMITED, Cambridge Cambridgeshire (GB)

(72) Inventors: Tuomas P. J. KNOWLES, Cambridge, Cambridgeshire (GB); Kadi Liis SAAR, Cambridge, Cambridgeshire (GB); Alekszej MORGUNOV, Cambridge, Cambridgeshire (GB)

(21) Appl. No.: 18/254,232

(22) PCT Filed: Oct. 26, 2021
(86) PCT No.: PCT/GB2021/052786

§ 371 Date: May 24, 2023

(30) Foreign Application Priority Data
Nov. 25, 2020 (GB) 2018514.6

(51) Int. Cl. G16B 15/30

(52) U.S. Cl. CPC G16B 15/30

(57) ABSTRACT

A computer implemented method of predicting liquid-liquid phase separation LLPS) behaviour of a biomolecule, the method comprising: inputting information identifying the biomolecule and its environmental composition and/or chemical modification of the biomolecule to an algorithm configured to predict, whether the biomolecule will exhibit LLPS under specified environmental conditions and/or chemical modification of the biomolecule, wherein: the algorithm is an algorithm generated by machine learning trained on data featurised according to features relating to biomolecules in the training data and features relating to the environmental conditions and/or chemical modification of the biomolecules in the training data, and the algorithm outputs a prediction of whether the biomolecule will exhibit LLPS under the specified environmental conditions and/or chemical modification of the biomolecule.

### TECHNICAL FIELD

The present disclosure relates to methods of predicting liquid-liquid phase separation behaviour in biomolecules.

### BACKGROUND ART

Liquid-liquid phase separation (LLPS) is a widely occurring biomolecular process that can lead to the formation of membraneless organelles within living cells. This process and the resulting condensate bodies are increasingly recognised to play an important role in a wide range of biological processes, including the onset and development of metabolic diseases and neurodegenerative disorders. Understanding how the LLPS behaviour of biomolecules, such as proteins or nucleic acids, can be influenced by environmental conditions, including the presence of other molecules, may be important for diagnosis and/or treatment of metabolic diseases and neurodegenerative disorders associated with condensate bodies.

Methods of predicting LLPS behaviour of proteins have been developed, such as the method disclosed in T. Sun, Q. Li, Y. Xu, Z. Zhang, L. Lai, and J. Pei, “Prediction of liquid-liquid phase separation proteins using machine learning,” bioRxiv, 2019. However, such methods are unable to provide information regarding how LLPS behaviour can be influenced by environmental conditions. Therefore such methods are limited in their use, e.g. for diagnosis and/or treatment of metabolic diseases and neurodegenerative disorders associated with condensate bodies.

It is an aim of the present disclosure to at least partially address some of the problems identified above.

### SUMMARY OF THE INVENTION

According to a first aspect of the invention there is provided a computer implemented method of predicting liquid-liquid phase separation (LLPS) behaviour of a biomolecule, the method comprising: inputting information identifying the biomolecule and its environmental composition and/or chemical modification of the biomolecule to an algorithm configured to predict whether the biomolecule will exhibit LLPS under specified environmental conditions and/or chemical modification of the biomolecule, wherein: the algorithm is an algorithm generated by machine learning, i.e. a machine learning algorithm, trained on data featurised according to features relating to biomolecules in the training data and features relating to the environmental conditions and/or chemical modification of the biomolecules in the training data, and the algorithm outputs a prediction of whether the biomolecule will exhibit LLPS under the specified environmental conditions and/or chemical modification of the biomolecule.

Optionally, the method comprises: inputting information identifying the biomolecule and its environmental composition to an algorithm configured to predict whether the biomolecule will exhibit LLPS under specified environmental conditions, wherein: the algorithm is a machine learning algorithm trained on data featurised according to features relating to biomolecules in the training data and features relating to the environmental conditions of the biomolecules in the training data, and the algorithm outputs a prediction of whether the biomolecule will exhibit LLPS under the specified environmental conditions.

Optionally, the method comprises: inputting information identifying the biomolecule and chemical modification of the biomolecule to an algorithm configured to predict whether the biomolecule will exhibit LLPS under specified chemical modification of the biomolecule, wherein: the algorithm is an algorithm generated by machine learning, i.e. a machine learning algorithm, trained on data featurised according to features relating to biomolecules in the training data and features relating to the chemical modification of the biomolecules in the training data, and the algorithm outputs a prediction of whether the biomolecule will exhibit LLPS under the specified chemical modification of the biomolecule.

Optionally, the method comprises: inputting information identifying the biomolecule, its environmental composition and chemical modification of the biomolecule to an algorithm configured to predict whether the biomolecule will exhibit LLPS under specified environmental conditions and chemical modification of the biomolecule, wherein: the algorithm is an algorithm generated by machine learning, i.e. a machine learning algorithm, trained on data featurised according to features relating to biomolecules in the training data and features relating to the environmental conditions and chemical modification of the biomolecules in the training data, and the algorithm outputs a prediction of whether the biomolecule will exhibit LLPS under the specified environmental conditions and chemical modification of the biomolecule.

Optionally, the biomolecule is a protein or a nucleic acid. Optionally, said prediction is carried out based on all or part of the amino acid sequence of said protein, or all or part of the nucleotide sequence of said nucleic acid and/or based on chemical substructures within the biomolecule.

Optionally, the specified environmental conditions comprise different levels of one or more types of environmental conditions, and the training data is featurised based on a level of the one or more types of environmental conditions.

Optionally, wherein at least one type of environmental conditions is selected from a plurality of possible types of environmental conditions.

Optionally, the types of environmental conditions include temperature, and the training data is featurised based on temperature.

Optionally, the types of environmental conditions include pH, and the training data is featurised based on pH.

Optionally, the types of environmental conditions include concentration of at least one chemical agent, and the training data is featurised based on a concentration of the at least one chemical agent.

Optionally, the training data is further featurised based on one or more properties of the at least one chemical agent in the environment of the protein. Optionally, the one or more properties include properties associated with one or more of: chemical composition, topology, and behaviour.

Optionally, the at least one chemical agent comprises nucleic acids, optionally polynucleotides or oligonucleotides comprising DNA and/or RNA. Optionally, the at least one chemical agent comprises a protein or a peptide. Optionally, the at least one chemical agent comprises a small molecule.

Optionally, the chemical modification of the biomolecule may be a biochemical modification. Optionally, the specified chemical modification comprises the presence of one or more types of chemical modification, and the training data is featurised based on a presence of the one or more types of chemical modification.

Optionally, the at least one type of chemical modification is selected from a plurality of possible types of chemical modification.

Optionally, the types of chemical modification include tagging with fluorescent tags, and the training data is featurised based on the presence of tagging with fluorescent tags.

When the types of chemical modification include tagging with fluorescent tags, the biomolecule may be a protein.

Optionally, the types of chemical modification include post-translational modifications, and the training data is featurised based on the presence of post-translational modifications.

When the types of chemical modification include post-translational modifications, the biomolecule is a protein.

Optionally, the types of chemical modification include fusion of the biomolecule with another biomolecule, and the training data is featurised based on the presence of fusion of the biomolecule with another biomolecule (optionally more than one other biomolecule). For example, where the biomolecule and the other biomolecule(s) are proteins, the biomolecule and the other biomolecule(s) may be fused together chemically after translation or made recombinantly as a single fusion protein encoded by a single coding DNA sequence.

Optionally, the training data is generated by systematic measurement of LLPS behaviour of biomolecules in varying environmental conditions.

Optionally, the training data is generated by systematic measurement of LLPS behaviour of biomolecules in varying states of chemical modification.

Optionally, the biomolecule is a protein or nucleic acid and the features of the biomolecule include the full amino acid or nucleic acid sequence.

Optionally, the biomolecule is a protein or a nucleic acid and the features of the biomolecule include the length of the amino acid or nucleotide sequence.

Optionally, the biomolecule is a protein and the features of the biomolecule include the hydrophobicity of the amino acid sequence.

Optionally, the biomolecule is a protein and the features of the biomolecule include the Shannon entropy of the amino acid sequence.

Optionally, the biomolecule is a protein and the features of the biomolecule include the fraction of low complexity regions of the amino acid sequence.

Optionally, the biomolecule is a protein and the features of the biomolecule include the fraction of intrinsically disordered regions of the amino acid sequence.

Optionally, the biomolecule is a protein and the features of the biomolecule include a fraction of polar, aromatic and/or cationic amino acid residues within low complexity regions of the amino acid sequence.

Optionally, the biomolecule is a protein or a nucleic acid, and the method comprises varying the amino acid or nucleotide sequence of said biomolecule to reflect the presence of mutations in the sequence and hence predict the LLPS behaviour of mutant forms of the biomolecule.

Optionally, the training data is separated into a plurality of distinct groups of biomolecules, based on propensity to exhibit LLPS. Optionally, the propensity to exhibit LLPS is, at least in part, based on the concentration at which the biomolecules exhibit LLPS, a relatively low concentration being associated with a relatively high propensity to exhibit LLP. Optionally, the biomolecule is a protein and the propensity to exhibit LLPS is, at least in part, based on the proportion of intrinsically disordered regions with the protein sequence, a relatively low proportion of intrinsically disordered regions being associated with relatively low propensity to exhibit LLPS.

According to a second aspect of the invention there is provided a method of identifying a biomolecule as a potential drug target, comprising applying the method of the first aspect to said biomolecule. Optionally, said biomolecule drug target is identified from among a plurality of potential targets. Optionally, the method comprises determining that the potential target is a biomolecule likely to exhibit a desired LLPS behaviour. Optionally, the method comprises determining that the potential target is a biomolecule likely to change LLPS behaviour in response to changes to environmental conditions. Optionally, the method comprises determining that the potential target is a biomolecule likely to change LLPS behaviour in response to chemical modification of the biomolecule.

According to a third aspect of the invention there is provided a method of identifying a potential therapeutic agent, comprising applying the method of the first aspect to a biomolecule, wherein said therapeutic agent is a chemical agent, as defined above in relation to the first aspect, in the environment of the biomolecule. Optionally, said biomolecule drug target is identified from among a plurality of potential targets.

Optionally, the method comprises determining that the therapeutic agent is a chemical agent that changes the LLPS behaviour of a biomolecule.

According to a fourth aspect of the invention there is provided a method of predicting whether LLPS behaviour of a biomolecule that is, or may be, associated with a disease may be present in a subject, based on measured environmental conditions within the subject, comprising applying the method of the first aspect to said biomolecule using said environmental conditions. Optionally, the method further comprises diagnosing the subject with said disease, and optionally treating said patient for said disease based on said diagnosis.

### BRIEF DESCRIPTION OF THE DRAWINGS

Further features of the invention are described below, based on non-limiting examples, with reference to the accompanying drawings in which:

FIG. 1 schematically shows an example method;

FIG. 2 shows experimentally acquired phase diagrams on the left, and predicted phase diagrams on the right, for temperature, temperature salt concentration, nucleic acid concentration, for four different proteins;

FIG. 3 shows t-distributed stochastic neighbouring embedding of the feature vectors describing individual experiments (blue/circles), and clusters (red/crosses) where phase separation occurred (left) or did not occur (right);

FIG. 4 shows predictions of the critical saturation concentration, with the experimentally observed values shown on the x-axis and the predicted value on the y-axis; performance of the model on the training data is shown on the left and on external test data on the right;

FIG. 5 shows two dimensional phase separation signature of HMGA1a compared to the rest of the proteome;

FIG. 6 shows phase separation of HMGA1 in the presence of DNA (left) and in the absence of DNA (right).

### DETAILED DESCRIPTION

The present disclosure provides a computer implemented method of predicting LLPS behaviour of a biomolecule under specified environmental conditions and/or chemical modification of the biomolecule. The biomolecule may any type of biomolecule that exhibits LLPS. For example, the biomolecule may be a protein or a nucleic acid. The method is performed based on an input identifying the biomolecule, for example, the amino acid sequence of a protein, or the nucleotide sequence of a nucleic acid. Alternatively, or additionally, this may be based on chemical substructures of the biomolecules. Optionally, one or more types of environmental conditions that are of interest, may also be provided as an input. This may be made as a selection from a plurality of possible selections of types of environmental conditions. Based on the inputs, an algorithm outputs a prediction of whether the protein will exhibit LLPS under the specified environmental conditions.

Optionally, one or more types of chemical modifications of the biomolecule that are of interest, may also be provided as an input. This may be made as a selection from a plurality of possible selections of types of chemical modifications.

Herein, a protein is understood to be a biomolecule comprising a sequence of amino acids of any length. A protein may thus be a short peptide, an oligopeptide, a polypeptide or a larger protein structure. A protein may have one or more domains or subunits. A protein will also typically comprise chemical substructures defined by some or all of inter- and/or intra-molecular bonds, non-covalent interactions, positive and/or negative charges, vibrational energies and other aspects of structure and/or chemistry. A protein may comprise entirely L amino acids as found in naturally occurring proteins, or a mixture of L and D amino acids, or entirely D amino acids. A protein may also include post-translational modifications, for example phosphorylation, glycosylation, ubiquitination, nitrosylation, methylation, acetylation, lipidation or SUMOylation.

Herein, a nucleic acid is understood to be a biomolecule comprising a sequence of nucleotides of any length. A nucleic acid may thus be an oligonucleotide, a polynucleotide, or a larger nucleic acid structure. For example, a nucleic acid may be a single-stranded or double-stranded molecule and may be linear or circular, for example an antisense oligonucleotide (AON), a small interfering RNA (siRNA), a short hairpin RNA (shRNA), a microRNA (miRNA), a CRISPR guide RNA, a plasmid or other circular DNA structure such as a viral genome, a messenger or transfer RNA or a chromosome or part thereof. A nucleic acid may consist of DNA, RNA or both DNA and RNA complex. A nucleic acid may comprise or consist of modified nucleotides, for example 2′-O-methoxyethylribose (MOE) modified nucleotides, locked nucleic acid (LNA) modified nucleotides or nucleoside phosphorothioates. A nucleic acid will also typically comprise chemical substructures defined by some or all of inter- and/or intra-molecular bonds, non-covalent interactions, positive and/or negative charges, vibrational energies and other aspects of structure and/or chemistry. In particular, RNA molecules, especially single-stranded RNAs, may have secondary structures such as stems of paired nucleic acids and loops of unpaired ones. A nucleic acid may be methylated in one or more positions, especially via methylation of cytosine moieties in DNA.

The methods of the invention can also be applied to complexes between protein molecules, between nucleic acid molecules and/or between protein and nucleic acid molecules, for example antibody/antigen complexes, DNA/transcription factor complexes, ribonucleoproteins or chromatin. Such complexes may be naturally occurring or artificial.

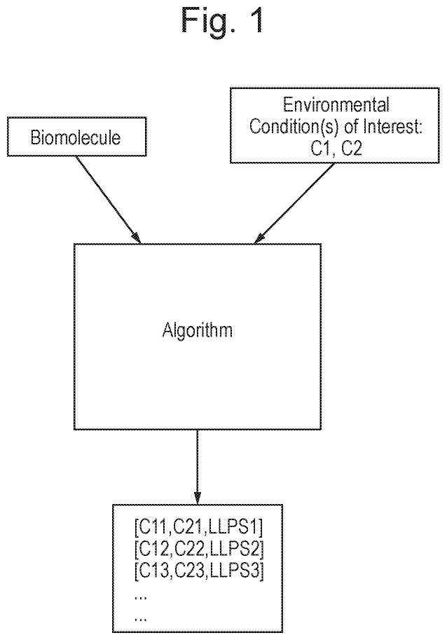

Such a method is schematically shown in FIG. 1. A first input identifies a biomolecule, a second input identifies environmental conditions of interest, C1 and C2 and the algorithm outputs vectors comprising combinations of different levels of conditions C1i and C2i, together with predicted LLPS behaviour, LLPSi for the given combination.

The output of the algorithm may include data comprising the specified environmental conditions and associated LLPS behaviour. The associated LLPS behaviour may be binary data, i.e. representing the presence of LLPS or the absence of LLPS. Behaviour may be predicted for different environmental conditions. Such data allows a predicted phase diagram to be constructed, that illustrates the LLPS behaviour of a biomolecule under the different environmental conditions. The data may be multi-dimensional, e.g. based on multiple different types of environmental conditions.

The inputs may comprise inputs identifying the presence of one or more chemical modifications of the biomolecule. These inputs may be included instead of or in addition to the inputs identifying environmental conditions. Then, the algorithm may output vectors comprising combinations of the presence of chemical modifications (and optionally environmental conditions) together with predicted LLPS behaviour for the given combination.

When the inputs comprise specified chemical modifications, the output of the algorithm may include data comprising specified chemical modifications and associated LLPS behaviour. The associated LLPS behaviour may be binary data, i.e. representing the presence of LLPS or the absence of LLPS. Behaviour may be predicted for different chemical modifications. Such data allows a predicted phase diagram to be constructed, that illustrates the LLPS behaviour of a biomolecule under the different chemical modifications. The data may be multi-dimensional, e.g. based on multiple different types of chemical modifications.

When the inputs comprise environmental conditions and chemical modifications, the output of the algorithm may include data comprising specified environmental conditions, specified chemical modifications, and associated LLPS behaviour. The associated LLPS behaviour may be binary data, i.e. representing the presence of LLPS or the absence of LLPS. Behaviour may be predicted for different environmental conditions and different chemical modifications. Such data allows a predicted phase diagram to be constructed, that illustrates the LLPS behaviour of a biomolecule under the different environmental conditions and chemical modifications. The data may be multi-dimensional, e.g. based on multiple different types of environmental modifications and chemical modifications.

Phase behaviour diagrams may be obtained in a bottom-up manner where the tendency of a biomolecule of interest to undergo phase behaviour is estimated under conditions of interest and the individual predictions are combined to construct a phase diagram. Alternatively, a model may be developed that directly learns the function that describes the boundary between a homogenous and a two-phased region on the phase diagram.

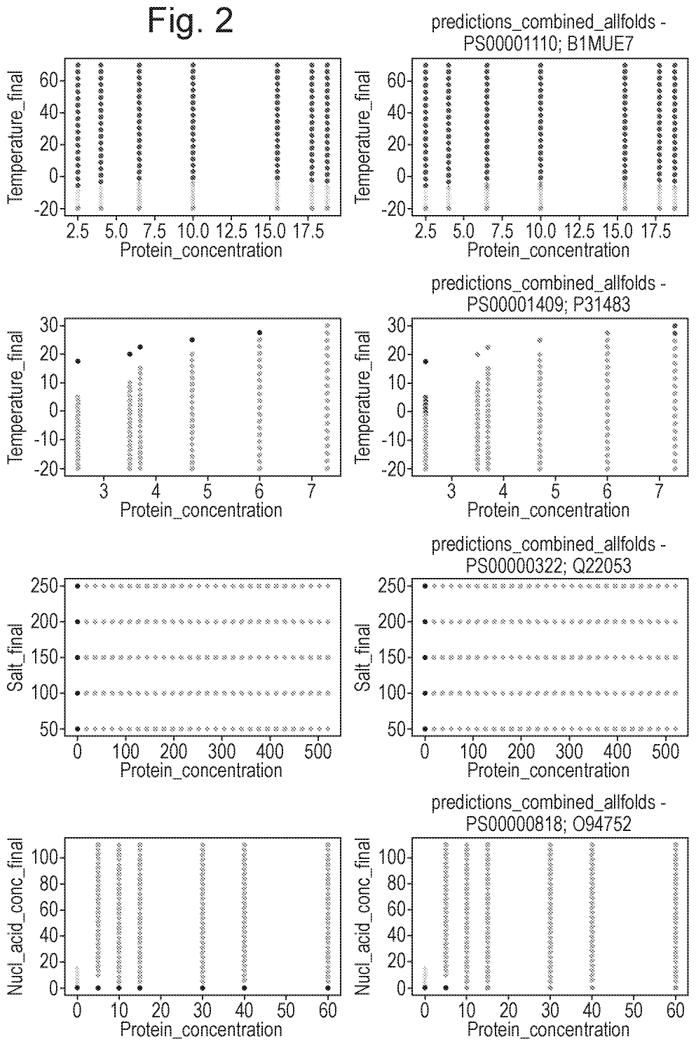

FIG. 2 show predicted phase diagrams output by an example algorithm. This shows how the method can predict the phase behaviour of proteins (left: experimentally acquired phase diagram, right: predicted phase diagram). Dark data points indicate no LLPS, light data points indicate LLPS. In all cases, the predictions are made on proteins that the model has not seen before. As is illustrated, predictions can be made on the effect various environmental changes, including temperature and salt concentration but also the inclusion of other molecules that modulate protein phase behaviour, such as nucleic acids.

The algorithm is built by machine learning (ML), i.e. it is a machine learning algorithm. The algorithm may be based on standard machine learning models, including regressors, SVM, tree-based classifiers, etc., as well as deep learning based models and neural networks. In an example method, the algorithm is a random forest classifier. The algorithm may be trained and validated in a typical way using the training data described below.

The algorithm is trained on data featurised according to features relating to biomolecules in the training data and features relating to the environmental conditions and/or chemical modification of the biomolecules in the training data.

The training data may be separated into a plurality of distinct groups of biomolecules, based on propensity to exhibit LLPS. The algorithm may be trained to differentiate between the distinct groups of biomolecules, i.e. the algorithm may be a classifier. The distinct groups of biomolecules may consist of two distinct groups of biomolecules, namely those with a relatively high propensity to exhibit LLPS and those with a relatively low propensity to exhibit LLPS. These two groups may be substantially disjoint, such that they are at substantially opposite ends on a scale of propensity to exhibit LLPS. Alternatively, the groups may be substantially adjacent, as oppose to disjoint.

The propensity to exhibit LLPS may, at least in part, be based on the concentration at which the biomolecules exhibit LLPS. A relatively low concentration may be associated with a relatively high propensity to exhibit LLPS. Accordingly, a data set of biomolecules with a relatively high propensity for LLPS may include biomolecules that exhibit LLPS at relatively low concentrations. In one example method, proteins that are observed exhibit LLPS for concentrations below 100 μM, on average are included in a high propensity data set. In one example method, proteins that are observed exhibit LLPS for concentrations above 100 μM are included in a low propensity data set, e.g. together with proteins that were not observed exhibit LLPS.

The concentration of the proteins, may not be the only indicator of propensity to exhibit LLPS. The propensity to exhibit LLPS may, at least in part, be based on the proportion of intrinsically disordered regions within the protein sequence. A relatively low proportion of intrinsically disordered regions may be associated with relatively low propensity to exhibit LLPS. In one example method, proteins that are not observed to exhibit LLPS and did not include any disordered amino acid residues are included in a low propensity data set.

Proteins included in the training data sets may optionally include proteins with only single naturally occurring protein construct, proteins with no post translational modifications, proteins with no repeat or single site mutations and/or proteins with a sequence longer than 50 amino acids. Nucleic acids included in the training data sets may optionally include nucleic acids with simple secondary structures, nucleic acids with no repeated sequences and/or nucleic acids with a sequence longer than 150 nucleic acids.

The data is featurised based on features relating to the biomolecule and features relating to the environmental conditions and/or chemical modification of the biomolecule. Features relating to proteins may include one or more of: the full amino acid sequence, the hydrophobicity of the amino acid sequence, the Shannon entropy of the amino acid sequence, the fraction of low complexity regions of the amino acid sequence, the fraction of intrinsically disordered regions of the amino acid sequence, a fraction of polar, aromatic, aliphatic, cationic and/or anionic amino acid residues within low complexity regions of the amino acid sequence, a fraction of a specific amino acid within low complexity regions of the amino acid sequence. Features relating to nucleic acids may include the full nucleic acid sequence, any secondary structure it possesses and/or its GC content.

The hydrophobicity of each of the protein sequence may be evaluated by summing the individual hydrophobicity values of the amino acids in the sequences using the Kyte and Doolittle hydropathy scale (J. Kyte and R. F. Doolittle, “A simple method for displaying the hydropathic character of a protein,” Journal of Molecular Biology, vol. 157, no. 1, pp. 105-132, 1982).

The Shannon entropy of each protein sequence may be estimated from formula 1, where p corresponds to the frequency of each of the naturally occurring twenty amino acid in the sequence.

$$H ( X ) = - {\sum }_{i = 0}^{N = 20} {p}_{i} {log}_{2} {p}_{i} \qquad ( 1 )$$

The low complexity regions (LCR) for each of the protein sequences may be estimated using the SEG Algorithm, e.g. with standard parameters (J. C. Wootton and S. Federhen, “Statistics of local complexity in amino acid sequences and sequence databases”, Computers & Chemistry, vol. 17, no. 2, pp. 149-163, 1993).

The disordered region may be predicted with UPred2a that estimates the probability of disorder for each of the individual amino acid residues in the sequence (Z. Dosztanyi, V. Csizmok, P. Tompa, and I. Simon, “The pairwise energy content estimated from amino acid composition discriminates between folded and intrinsically unstructured proteins,” Journal of Molecular Biology, vol. 347, no. 4, pp. 827-839, 2005). The disorder fraction of a sequence may be calculated as the fraction of residues in the total sequence that were considered disordered. A specific residue may be classified as disordered when the disorder probability stays above 0.5 for at least 20 consecutive residues.

The amino acid sequence and the LCR regions may be described for their amino acid content by allocating the residues to the following groups: amino acids with polar residues (Serine, Glutamine, Asparagine, Glycine, Cysteine, Threonine, Proline), with hydrophobic residues (Alanine, Isoleucine, Leucine, Methionine, Phenylalanine, Valine), with aromatic residues (Tryptophan, Tyrosine, Phenylalanine), with cationic residues (Lysine, Arginine, Histidine) and with anionic residues (Aspartic acid, Glutamic acid).

A biomolecule sequence may be featurised based on unsupervised sequence embeddings. The sequence embeddings may be generated by pre-training performed on a data set of biomolecules. For example, a word2vec skip-gram pre-training procedure may be used.

In one example method, with the assistance of Python gensim library, 200-dimensional embedding vectors are generated. The full Swiss-Prot database (accessed on 26 Jun. 2020) using 3-grams, a window size of 25 and negative sampling is used. This process results in 200-dimensional embedding vectors for each of the protein sequences that served as the input features when training the machine learning classifiers. Each protein sequence used for training the algorithm may be broken into 3-grams using all three possible reading frames. The final 200-dimensional full protein embedding may be obtained by summing all constituent 3-gram embeddings.

The chemical modifications may be featurised based on different types of chemical modification including one or more of: fluorescent tagging/labelling of the biomolecule, post-translational modifications of the biomolecule, and fusion of the biomolecule with another biomolecule. These chemical modifications may be featurised using a numerical input or by being assigned to a category.

The environmental conditions may be featurised based on different types of environmental conditions including one or more of: temperature, pH, salt concentration, and concentration of a chemical agent.

Environmental conditions such as temperature, pH or salt concentration may be featurised using a numerical input or assigned to a category, with thresholds used to divide the data into a discrete number of categories.

Chemical agents may be featurised according to one or more of concentration, an identity of the chemical agent, and a property of the chemical agent. The properties may be associated with one or more of: chemical composition, topology, and behaviour of the chemical agent, for example.

The chemical agents may include nucleic acids, such as polynucleotides and oligonucleotides comprising DNA and/or RNA. For example, these may be featurised based on one or more of:
explicit features describing the sequence composition (e.g. abundance of various bases, frequency of different n-grams or motifs, parameters describing biases in sequence distribution, such as Shannon entropy).
explicit features extracted from structure (proximity of various bases to each other, the precise secondary structure element each base is part of).
implicit distribution semantics governed embedding algorithms (e.g. word2vec, doc2vec).
Recurrent neural networks based algorithms, e.g. “vanilla” RNN, GRU, LSTM, biLSTM, ELMo
Self-attention network based algorithms, e.g. transformers, derivatives, e.g. BERT, XL-Net

The chemical agents may include proteins and peptides. For example, these may be featurised based on one or more of:
explicit features describing the sequence composition (abundance of various amino acids and their type (polar, hydrophobic, anionic, cationic, aromatic, aliphatic etc), frequency of different n-grams or motifs, parameters describing biases in sequence distribution, such as Shannon entropy).
explicit features derived from the sequence, such as charge, hydrophobicity, degree of intrinsic disorder either locally (for a short segment of the sequence) or across the full sequence.
explicit features extracted from the structure (proximity of various amino acids to each other, the precise secondary structure element each residue is part of).
implicit distribution semantics governed embedding algorithms (e.g. word2vec, doc2vec).
Recurrent neural networks based algorithms, e.g. “vanilla” RNN, GRU, LSTM, biLSTM, ELMo (e.g. SeqVec)
Self-attention network based algorithms, e.g. transformers, derivatives, e.g. BERT, XL-Net (e.g. ProtTrans)

The chemical agents may include small molecules. For example, these may be featurised based on one or more of:
features governing the abundance of different atoms and/or chemical connectivity between atoms.
the topology of the molecule.
3D coordinates of the atoms, including their variation in time, e.g. as can be extracted from molecular dynamics simulations.
Features that can be extracted through other characterisation techniques, such as shifts in NMR spectra.
Variational autoencoders and graph-neural networks.

Prior to training a machine learning model, data augmentation and data sampling steps may be used to ensure generalisability of the trained model.

For example, physical insight into how chemical modifications affect phase behaviour may be used to augment existing data.

Specifically, physical insight into how changes in environmental conditions affect phase behaviour may be used to augment existing data. For example, when phase separation is known to occur at a specific biomolecule concentration, the training data may be augmented with points that describe the tendency of the specific biomolecule to undergo phase separation at all other conditions on the tie line or within the area surrounded by the phase separation binodal.

Additionally, available data may be selectively sampled to reduce the probability that the model becomes overfitted but instead remains generalisable. This may be achieved by estimating relative similarity between individual data points in the training data set.

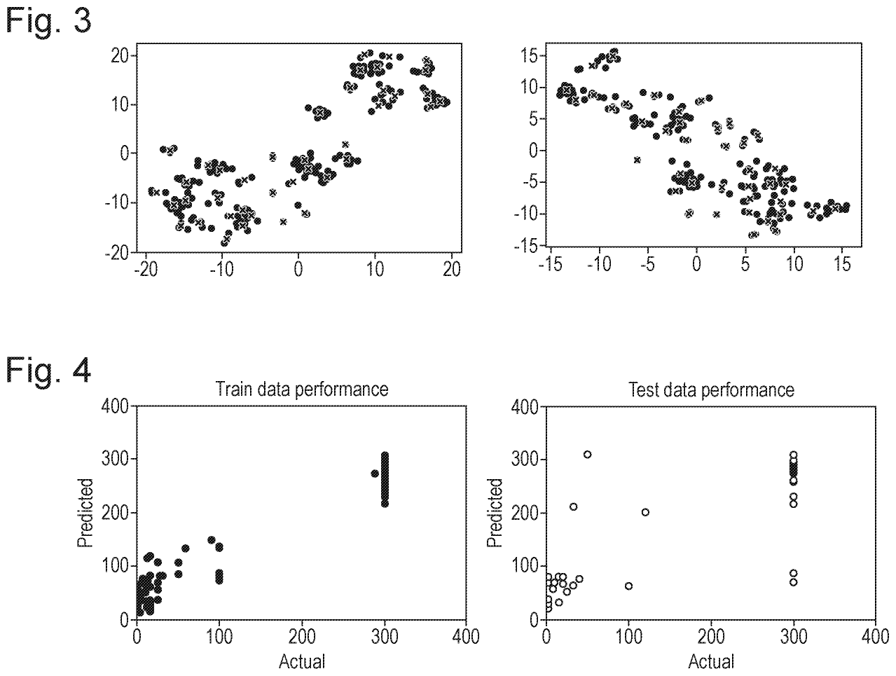

For example, FIG. 3 shows a t-distributed stochastic neighbouring embedding of the data points under which phase separation occurred (left) or did not occur (right). Here, the vectors describing the individual data points involved contributions from the fraction of aromatics, cations, hydrophobicity, Shannon entropy, LCR fraction, IDR fraction, isoelectric point, salt concentration, protein concentration, cumulative crowding agent concentration and cumulative hydrophobic additive concentration (circles). In general, any featurisation approach may be used. The data points were then clustered using a K-mean clustering algorithm with a known number of clusters (crosses). Other approaches for dimensionality reduction, such as principal component analysis or uniform manifold approximation and projection may be used to estimate their similarity in a low-dimensional space. Alternatively, metrices such as Euclidian distance or Manhattan distance may be used to describe the similarity between individual points. For training, only one point per cluster may be chosen. Moreover, a number of different models may be trained with a single point sampled from each cluster. In this latter case, the final prediction may be based on an ensemble of the trained models.

Using this or other training strategies, biomolecule phase behaviour may be predicted. To this effect, one possibility involves predicting the critical concentration at which a biomolecule undergoes phase behaviour (cₛₐₜ). For example, FIG. 4 shows the predictions made on cₛₐₜ using a random forest regressor model. Here, a combination of features that is estimated directly from the amino acid sequence of a protein and distributional semantics based embeddings were used to describe each protein sequence. Sequences that had not been observed to undergo phase separation experimentally were given a high concentration at which they would phase separate. The predictions that the example trained model made on the training data are shown in the left and on performance on test data on the right with the actual concentration shown on the x-axis and the predicted value on the y-axis. The training and the test data did not include any similar sequences, e.g. that would share a UniProt ID. Data may be split between training and test data, or training and validation data, such that sequences with the same UniProt ID are in the same group. In general, the split between training and test data may either be performed randomly or by using a specific constraint on grouping, e.g. by ensuring that sequences with the same Uniprot ID or those that share sequence similarity above a specific threshold would always be in the same group (train or test data). Alternative network structures may be used and approaches may be used for training. For example, a multitask learning process may be performed where a regressor model is built on top of or in parallel with a binary prediction of the phase behaviour.

After the critical saturation concentrations (cₛₐₜ) have been estimated, phase diagrams may be constructed applying physical insight about how the system behaves at concentrations above or below the critical concentration.

Alternatively, phase diagrams may be predicted directly by either estimating phase behaviour at various points across a multidimensional phase diagram or predicting the boundary between a homogenous mixed region and a two-phased demixed region. The predicted phase diagrams estimated using this strategy are shown in FIG. 2

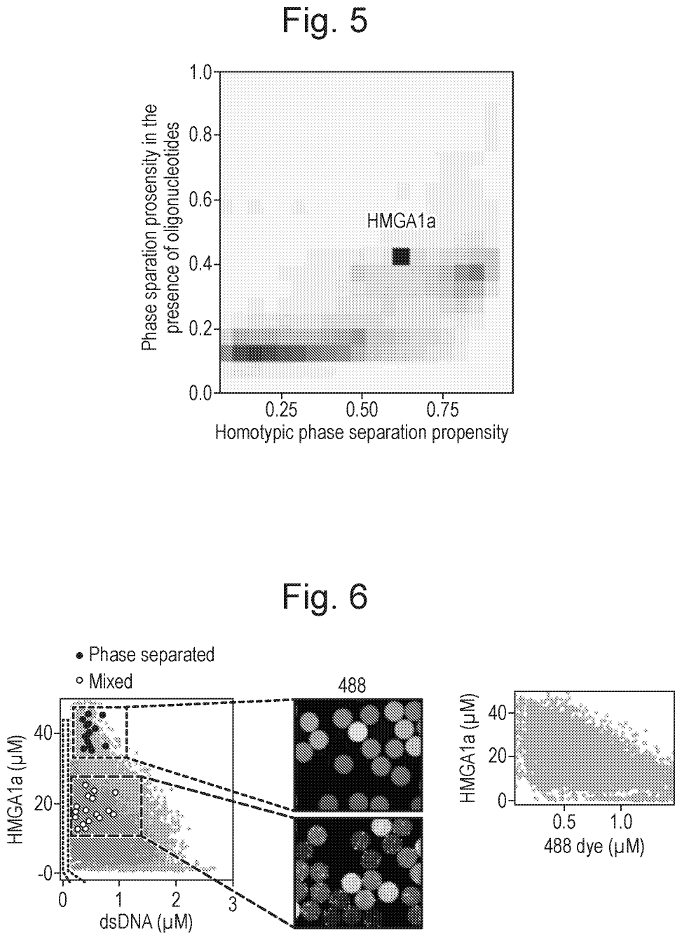

FIG. 5 shows the two dimensional phase separation signature of HMGA1a predicted by an example method. Compared to the rest of the proteome, HMGA1 stands out as a protein whose phase behaviour is strongly promoted by oligonucletodes. Specifically, in order to evaluate if the presence of oligonucleotides enhances the phase separation propensity of specific proteins, we obtain the two dimensional phase separation signature of each protein of interest.

The signature describes the propensity of a molecule undergo phase separation in a homotypic environment and in the presence of oligonucleotides. This signature estimation involves training two parallel machine learning algorithms, one that is trained to evaluate the propensity of the protein to undergo phase separation in a homotypic environment and the other one is based on estimating the propensity of a protein to co-localise with RNA-rich condensates. In the former case, the protein is featurised through a combination of a pre-trained embeddings (also known as protein Language Model) and specifically engineered features derived directly from the sequence. In the latter case, the featurisation of the protein is performed by using both of these features (pre-trained embeddings and specifically engineered features derived directly from the sequence) in combination with information that is available from various biomolecular databases, such as its interactions with other proteins (available from StringDB and BIOGRID) and with RNA and DNA.

The two-dimensional protein separation signature suggested that in comparison to all other proteins in the human proteome, the phase separation propensity of HMGA1 is heavily increased by the introduction of oligonucleotides (FIG. 5), suggesting oligonucleotides are important components for the promotion the phase separation of HMGA. This prediction was tested experimentally by analysing HMGA1. It was noticed that HMGA1 did not form condensates (FIG. 6, right) even at concentrations as high as 40 μM. However, when DNA was added, phase separated droplets appeared even as low as nanomolar concentrations (FIG. 6, left), demonstrating that DNA is an important component for triggering the phase separation process. This example illustrates how a single-value based metric is insufficient to capture the full nuance of the phase separation behaviour and motivates the introduction of environment specific predictions as is described above.

In addition to capturing the effect of chemical changes in the environment, such as a concentration of oligonucleotides (shown in FIGS. 5 and 6) or salt (shown in FIG. 2), the biological context and the form of the protein that gets expressed under specific circumstances can play a strong effect on whether a protein phase separates. As an example, the above disclosed methods suggest that HOXA9 (a transcription factor based protein) on its own has only a mild propensity to form condensates (score of 0.62). However, its modified version where it is fused to NUP98, is predicted to have a score of 0.85, suggesting an increased phase separation propensity upon the fusion event. Experimentally, it has indeed been seen that in patients with leukemia where NUP98-HOXA9 fusions occur, condensates are frequently detected and their occurrence has been linked to the onset of cancer (Ahn, J. H., Davis, E. S., Daugird, T. A. et al. Phase separation drives aberrant chromatin looping and cancer development. Nature 595, 591-595 (2021). https://doi.org/10.1038/s41586-021-03662-5; https://www.nature.com/articles/s41586-021-03662-5).

As an additional example, the algorithm can be used to identify if modifications to a protein sequence, such as their tagging with fluorescent tags allows us to correctly predict the effect of these modifications to protein phase behaviour. To this effect, we have investigated the sequences from Mohan et al. (Mohan, K V K et al. “The N- and C-terminal regions of rotavirus NSP5 are the critical determinants for the formation of viroplasm-like structures independent of NSP2.” Journal of virology vol. 77, 22 (2003): 12184-92. doi:10.1128/jvi.77.22.12184-12192.2003;
https://www.ncbi.nlm.nih.gov/pmc/articles/PMC254265/) where the researchers found that the introduction of a GFP-tag to NSP5 protein results in the protein turning from a non phase-separating protein to a phase separating one which is in agreement with our predictive algorithm (increase from 0.61 to 0.82). This highlights another potential advantage of the developed algorithms—the capability to estimate the effect of biochemical modifications, such as labelling.

An additional example of how the algorithm can be used to predict the effect of chemical modifications is related to its capability to predict the effect of post-translational modifications. This capability is achieved by replacing modified amino acids with their relevant mimetics, such as the introduction of aspergic acid uponphosphorylation. This is a potential advantage of the algorithm because under cellular conditions, proteins are often in post-translationally modified states.

Owen et al have recently reviewed how post-translational modifications affect protein phase behaviour (Owen I, Shewmaker F. The Role of Post-Translational Modifications in the Phase Transitions of Intrinsically Disordered Proteins. Int J Mol Sci. 2019; 20(21):5501. Published 2019 Nov. 5. doi:10.3390/ijms20215501;
https://www.ncbi.nlm.nih.gov/pmc/articles/PMC6861982/). When applying the algorithm to predict if phosphorylation affects the phase separation propensity, correct predictions were obtained both for the cases when the chemical modification had been experimentally observed to increase (FMRP and Tau proteins) or to decrease (FUS, TDP-43) the phase separation propensity.

### First Example Application

A first key area of application is drug target identification. Out of the myriad of biomolecules whose expression level changes with the onset or the progression of a disease, the disclosed models can identify the molecules that are the most likely to undergo phase transition and serve as the most suitable targets for drug development.

The disclosed models give a further possibility to predict how biomolecular phase behaviour is affected by changes in nucleotide or amino sequence composition. The models may be used to identify which molecules have their phase behaviour most susceptible to alterations of their sequence composition, in particular by mutations that occur frequently as part of the diseases of interest, or that might occur. For example, either the effects of known mutations at given positions in a biomolecule or those of postulated mutations at any selected position can be modelled.

Moreover, many key biomolecular targets that are believed to be central to the onset of progression of pathological conditions have remained “undruggable” by drug development campaigns performed to date. However, these targets can become accessible upon a transition from a soluble form to a fully condensed state. In particular, many intrinsically disordered proteins that do not present well-defined “pockets” into which drugs can bind may become targetable when they have transitioned into a condensed form.

The above described method of predicting LLPS behaviour of a biomolecule under specified environmental conditions may be used in a method of identifying a drug target, from among a plurality of potential targets. The method may comprise determining that the target is a biomolecule likely to exhibit a desired LLPS behaviour. The method may comprise determining that the target is a biomolecule likely to change LLPS behaviour in response to changes to environmental conditions. The method may comprise screening the plurality of potential targets using the algorithm.

### Second Example Application

In addition to estimating whether a certain biomolecule is likely to undergo phase transition or not, the disclosed models allow predicting how the phase behaviour is affected by changes in environmental conditions, including their modulation by chemical entities, e.g. drug candidates. This opens up the second key area of applications, which is the identification of drug molecules that affect the phase behaviour of identified targets.

Data from high-throughput experimental screens in combination with biomolecular representation approaches to develop models for predicting how changes in environmental conditions, including the introduction of various chemical entities, affects and modulates biomolecular phase behaviour.

In parallel, the predictive models serve as the first pre-filtering step for narrowing down the list of drug candidates that needs to be screened experimentally.

Overall, the combination of predictive ML models and high-throughput screening approaches will provide a platform for active learning to identify promising drug candidates both from existing libraries and through the use of generative chemical models.

The above described method of predicting LLPS behaviour of a biomolecule under specified environmental conditions may be used in a method of identifying a therapeutic agent, from among a plurality of potential therapeutic agents. The method may comprise determining that the therapeutic agent is a chemical agent that changes the LLPS behaviour of a biomolecule. The method may comprise screening the plurality of potential therapeutic agents using the algorithm.

### Third Example Application

Disease diagnosis through analysing the condensate landscape of a patient. The disclosed models of biomolecular phase behaviour can be combined with experimental tools that profile the condensomic landscape of a patient.

The predictive models may be used to link the condensomic landscape to potential onset of a disease or make prognosis about its progression.

Using data on the condensomic landscape of specific patients, such as the biomolecular composition of the condensates, the models allow predictions of what other molecules may get integrated with these condensates next and what wider disease implications this integration step may have.

The above described method of predicting LLPS behaviour of a biomolecule under specified environmental conditions may be used in a method of predicting whether LLPS behaviour of a biomolecule that is associated with a disease may be present in a subject, based on measured environmental conditions within the subject. The method may comprise diagnosing the subject with said disease.

### Fourth Example Application

An additional area where the predictive models are used is the production or synthesis of biochemical molecules, including but not being limited to drug candidates. With condensate formation providing a possibility to spatially control cellular arrangement of molecules, this phenomenon can be exploited to modulate the activity of enzymes of interest that through up- or downregulation can result in increased production of a chemical of interest. The disclosed models can be used to predict the optimum conditions for the production of a variety of biomedical compounds.

Variations of the above described examples are possible in light of the above teachings. It is to be understood that the invention may be practised otherwise than specifically described herein without departing from the spirit and scope of the invention.

1. A computer implemented method of predicting liquid-liquid phase separation (LLPS) behaviour of a biomolecule, the method comprising:
inputting information identifying the biomolecule and its environmental composition and/or chemical modification of the biomolecule to an algorithm configured to predict, whether the biomolecule will exhibit LLPS under specified environmental conditions and/or chemical modification of the biomolecule, wherein:
the algorithm is a machine learning algorithm trained on data featurised according to features relating to biomolecules in the training data and features relating to the environmental conditions and/or chemical modification of the biomolecules in the training data, and
the algorithm outputs a prediction of whether the biomolecule will exhibit LLPS under the specified environmental conditions and/or chemical modification of the biomolecule.

2. The method of claim 1, wherein the biomolecule is a protein or a nucleic acid.

3. The method of claim 2, wherein said prediction is carried out based on all or part of the amino acid sequence of said protein, or all or part of the nucleotide sequence of said nucleic acid and/or based on chemical substructures within the biomolecule.

4. The method of any preceding claim, wherein the specified environmental conditions comprise different levels of one or more types of environmental conditions, and the training data is featurised based on a level of the one or more types of environmental conditions.

5. The method of claim 4 wherein at least one type of environmental conditions is selected from a plurality of possible types of environmental conditions.

6. The method of any one of claim 4 or 5, wherein the types of environmental conditions include temperature, and the training data is featurised based on temperature.

7. The method of any one of claims 4 to 6, wherein the types of environmental conditions include pH, and the training data is featurised based on pH.

8. The method of any one of claims 4 to 7, wherein the types of environmental conditions include concentration of at least one chemical agent, and the training data is featurised based on a concentration of the at least one chemical agent.

9. The method of claim 8, wherein the training data is further featurised based on one or more properties of the at least one chemical agent.

10. The method of claim 9, wherein the one or more properties include properties associated with one or more of: chemical composition, topology, and behaviour.

11. The method of claim 10, wherein the at least one chemical agent comprises nucleic acids, optionally polynucleotides or oligonucleotides comprising DNA and/or RNA.

12. The method of claim 10, wherein the at least one chemical agent comprises a protein or a peptide.

13. The method of claim 10, wherein the at least one chemical agent comprises a small molecule.

14. The method of any preceding claim, wherein at least one type of chemical modification is selected from a plurality of possible types of chemical modification.

15. The method of claim 14, wherein the types of chemical modification include tagging with fluorescent tags, and the training data is featurised based on tagging with fluorescent tags.

16. The method of claim 14 or 15, wherein the types of chemical modification include post-translational modifications, and the training data is featurised based on post-translational modifications.

17. The method of any one of claims 14 to 16, wherein the types of chemical modification include fusion of the biomolecule with another biomolecule, and the training data is featurised based on the fusion of the biomolecule with another biomolecule.

18. The method of any preceding claims, wherein the training data is generated by systematic measurement of LLPS behaviour of biomolecules in varying environmental conditions and/or in varying chemical modifications.

19. The method of any preceding claim, wherein the biomolecule is a protein or nucleic acid and the features of the biomolecule include the full amino acid or nucleic acid sequence.

20. The method of any preceding claim, wherein the biomolecule is a protein or a nucleic acid and the features of the biomolecule include the length of the amino acid or nucleotide sequence.

21. The method of any preceding claim, wherein the biomolecule is a protein and the features of the biomolecule include the hydrophobicity of the amino acid sequence.

22. The method of any preceding claim, wherein the biomolecule is a protein and the features of the biomolecule include the Shannon entropy of the amino acid sequence.

23. The method of any preceding claim, wherein the biomolecule is a protein and the features of the biomolecule include the fraction of low complexity regions of the amino acid sequence.

24. The method of any preceding claim, wherein the biomolecule is a protein and the features of the biomolecule include the fraction of intrinsically disordered regions of the amino acid sequence.

25. The method of any preceding claim, wherein the biomolecule is a protein and the features of the biomolecule include a fraction of polar, aromatic and/or cationic amino acid residues within low complexity regions of the amino acid sequence.

26. The method of any preceding claim, wherein the biomolecule is a protein or a nucleic acid, the method comprising varying the amino acid or nucleotide sequence of said biomolecule to reflect the presence of mutations in the sequence and hence predict the LLPS behaviour of mutant forms of the biomolecule.

27. The method of any preceding claims, wherein the training data is separated into a plurality of distinct groups of biomolecules, based on propensity to exhibit LLPS.

28. The method of claim 27, wherein the propensity to exhibit LLPS is, at least in part, based on the concentration at which the biomolecules exhibit LLPS, a relatively low concentration being associated with a relatively high propensity to exhibit LLPS.

29. The method of claim 27 or 28, wherein the biomolecule is a protein and the propensity to exhibit LLPS is, at least in part, based on the proportion of intrinsically disordered regions with the protein sequence, a relatively low proportion of intrinsically disordered regions being associated with relatively low propensity to exhibit LLPS.

30. A method of identifying a biomolecule as a potential drug target, comprising applying the method of any preceding claim to said biomolecule.

31. The method of claim 30, wherein said biomolecule drug target is identified from among a plurality of potential targets.

32. The method of claim 28, comprising determining that the potential target is a biomolecule likely to exhibit a desired LLPS behaviour.

33. The method of claim 31 or 32, comprising determining that the potential target is a biomolecule likely to change LLPS behaviour in response to changes to environmental conditions and/or chemical modification.

34. A method of identifying a potential therapeutic agent, comprising applying the method of any one of claims 1 to 27 to a biomolecule, wherein said therapeutic agent is a chemical agent, as defined in any one of claims 7 to 12, in the environment of the biomolecule.

35. The method of claim 34, wherein said biomolecule drug target is identified from among a plurality of potential targets.

36. The method of claim 34, comprising determining that the therapeutic agent is a chemical agent that changes the LLPS behaviour of a biomolecule.

37. A method of predicting whether LLPS behaviour of a biomolecule that is, or may be, associated with a disease may be present in a subject, based on measured environmental conditions within the subject, comprising applying the method of any one of claims 1 to 27 to said biomolecule using said environmental conditions.

38. The method of claim 37, further comprising diagnosing the subject with said disease, and optionally treating said patient for said disease based on said diagnosis.
